(19) United States
(12) Patent Application Publication
(10) Pub. No.: US 2019/0175206 A1
(43) Pub. Date: Jun. 13, 2019

(54) METHOD AND DEVICES FOR USING VIBRATIONAL ENERGY FOR CARDIAC LEAD REMOVAL

(71) Applicant: Justin Panian, San Francisco, CA (US)

(72) Inventor: Justin Panian, San Francisco, CA (US)

(21) Appl. No.: 16/144,915

(22) Filed: Sep. 27, 2018

Related U.S. Application Data
(63) Continuation-in-part of application No. 15/981,083, filed on May 16, 2018, now abandoned.
Application No. 15/981,083 is a continuation of application No. 15/904,108, filed on Feb. 23, 2018, now abandoned.
(60) Provisional application No. 62/596,209, filed on Dec. 8, 2017.

(51) Int. Cl. A61B 17/32; A61N 1/05

(52) U.S. Cl. CPC A61B 17/320068; A61B 2017/00477; A61B 2017/320078; A61N 1/0587

(57) ABSTRACT

A method and devices are provided for separating cardiac lead from encapsulating tissue in a patient. The device includes an elongated shaft with a passageway that allows the lead and any tissue remaining or attached to the lead to enter the device. The device includes a vibrating tip positioned at the distal end of the elongated shaft to ablate and separate tissue attached to leads, and to facilitate lead removal.

### FIELD OF INVENTION

The present invention relates generally to methods and devices for use in separating an implanted cardiac lead from encapsulating tissue in the body of a patient. More particularly, the invention relates to devices with a vibrating tip/ring for ablating and separating tissue attached to leads, and for removing leads.

### BACKGROUND OF THE INVENTION

Cardiac rhythm disorders such as atrial fibrillation, tachycardia or sudden cardiac arrest are often treated by stimulation therapy using cardiac pacing. Cardiac pacing systems typically include a timing device and a lead, which are placed inside the body of a patient.

One part of the system is the pulse generator containing electric circuits and a battery, usually placed under the skin on the wall of the chest beneath the collarbone. Another part of the system includes the wires, or leads, which run between the pulse generator and the heart.

In a pacemaker, these leads allow the device to increase the heart rate by delivering small timed bursts of electric energy to make the heart beat faster. In a defibrillator, the lead has special coils to allow the device to deliver a high-energy shock and convert potentially dangerous rapid rhythms (ventricular tachycardia or fibrillation) back to a normal rhythm. Additionally, the leads may transmit information about the heart's electrical activity to the pacemaker.

Cardiac pacemakers are typically implanted in a subcutaneous tissue pocket in the chest wall of a patient. A pacemaker lead extends from the pacemaker through a vein into a chamber of the patient's heart. The pacemaker lead commonly includes a conductor, such as an electrical wire/coil, for conducting electrical signals (such as stimulating and/or sensing signals) between the pacemaker and the heart. Leads for defibrillators are generally similar to pacemaker leads, and are positioned about the heart. Defibrillator leads may be affixed either internally or externally of the heart.

Within a relatively short time after a lead is implanted into the body, the body's natural healing process forms scar tissue and often calcifications along the lead. Although leads are designed to be implanted permanently in the body, over time these leads must be removed, or extracted due to numerous reasons, including but not limited to, infections, lead age, and lead malfunction. Therefore, removal of lead may be desirable and often necessary.

Removing an electrical lead from the heart, however, is not only expensive and time consuming, but poses numerous risks to the patient, such as injury to cardiac tissue and excessive bleeding. To avoid possible complications, some useless or otherwise inoperable cardiac leads are simply left in the patient when the pacemaker or defibrillator is removed or replaced. However, such a practice can incur the risk of an undetected lead thrombosis, which can result in stroke, heart attack, pulmonary embolism or infections.

Surgical removal of a heart lead may require open heart surgery which is accompanied by significant risk and cost to the patient, as well as a potential for unintended complications.

A variety of methods and apparatuses have been proposed as alternatives to open heart surgeries, and describe manual or mechanical devices that are used for removing an implanted cardiac lead. Others describe non-mechanical techniques, such as laser extraction or radio-frequency extraction.

Ultrasound energy has also been also proposed to remove leads. U.S. Pat. No. 6,241,691 (Tu et al.) discloses the use of a catheter with an ultrasound transducer tip where the transducer is located at the distal end of the catheter to ablate tissue at high frequencies above 1 MHz.

While the prior art devices have been found to be reasonably effective in removing leads surrounded by soft or scarred tissue, however, calcifications of the lead encapsulating tissue continue to be a challenge. It is difficult to cut calcified tissue by mechanical means such as rotating cutters or lasers. Ultrasound energy at frequencies above 100 kHz is also ineffective due to very small tip displacement and the creation of heat. In addition, lead removal using these less-invasive and non-surgical methods lacks a suitable or direct visualization when a lead is being separated or cut from a tissue. Even if the lead removal procedure is performed under x-ray, it is an intuitive approach without clearly seeing exactly what is being cut.

Considering the fact that almost 50% of the lead encapsulating tissue involves hardened tissue and calcifications, in some cases, physicians encounter particularly challenging myocardial perforations while removing lead, often causing internal bleedings. If such bleeding is not addressed quickly by a cardiosurgical team, it may result in patient death. Since the less-invasive procedures are typically performed by a cardiologist, handling such life-threatening myocardial perforation emergencies is outside of their skills and expertise, and require surgical repair. Also, patients with lead encapsulating calcific tissue must often undergo surgical procedure.

Many more lead removal procedures would be performed using less-invasive methods rather than surgery if new devices can safely ablate or separate lead encapsulating calcific tissue to further minimize or eliminate risk of myocardial perforations. Most of the less-invasive lead removal procedures are performed by a cardiologist, and handling such life-threatening myocardial perforation emergencies is outside of their expertise and require surgical repair.

Accordingly, there is a need for devices and methods that are capable of ablating, cutting or separating lead from the encapsulating calcific tissue while maintaining safety to a soft and healthy surrounding tissue.

### SUMMARY OF THE INVENTION

Devices of the present invention to extract leads comprise a sheath assembly having a coaxial flexible tube that passes over the lead and/or the surrounding tissue. A cutting ring/tip is attached to the distal end of the sheath that may be vibrated and is aligned coaxially with the sheath. Upon advancement, the tip/ring cuts or separates the lead encapsulating tissue.

When the lead is encapsulated by soft tissue, the device of the present invention may be pushed distally around the lead to cut encapsulating tissue. In case the lead is encapsulated in a hardened calcific tissue, vibrational energy may be used to vibrate the ring/tip to further ablate calcifications and to facilitate lead separation from the encapsulating tissue. Once the lead is separated from the encapsulating tissue, it may be pulled by itself or by using a locking stylet outside the patient.

The vibrating cutting tip/ring of the present invention is capable of ablating hard tissue and calcifications, while providing safety to healthy tissue and promoting elasticity of soft tissue. This unique feature of using a vibrating cutting tip/ring for selective calcific tissue ablation, while leaving intact a healthy tissue, is utilized to separate leads from encapsulating tissue and may be helpful in reducing and minimizing myocardial perforations. If the vibrating cutting tip/ring faces a soft healthy myocardial tissue, it will not cut that tissue while still being capable of cutting and ablating hardened or calcific tissue that is encapsulating the lead.

Given the rising number of implanted transvenous pacemakers, ICDs, and CRT, transvenous lead extractions are going to be performed more frequently. To ensure safety and success, it is imperative to have better and safer devices, given the technical complexity and the risk of life-threatening complications that can arise. Old leads, calcifications, infections, and multiple leads make the procedure technically more difficult. Therefore, simplification of the lead removal procedure is another major objective of the present invention to avoid major complications and to ensure higher success rates.

“Vibrations” and ‘vibrational energy” have the same meaning and refer to physical vibrations of the tip/ring at the frequency range of 1 Hz to less than 1 MHz.

### BRIEF DESCRIPTION OF DRAWINGS

FIG. 1 illustrates a lead extraction device according to one embodiment of the present invention.

FIG. 2 shows an alternative version of the lead extraction device with an inner sheath.

FIG. 3 illustrates the lead extraction device of FIG. 1 introduced over the lead and positioned proximal to tissue that has encapsulated the lead.

FIG. 4 illustrates the lead extraction device positioned distal to the encapsulating tissue, and the lead separated from encapsulating tissue to be retrieved from the body.

FIG. 5 illustrates the ultrasound energy source as shown in FIGS. 1 and FIG. 3.

FIG. 6 illustrates the ultrasound energy source as shown in FIG. 2.

### DETAILED DESCRIPTION OF DRAWINGS

The vibrational devices of the present invention are insertable into the human body and usable to deliver vibrational energy for the purpose of ablating obstructive material encapsulating cardiac leads.

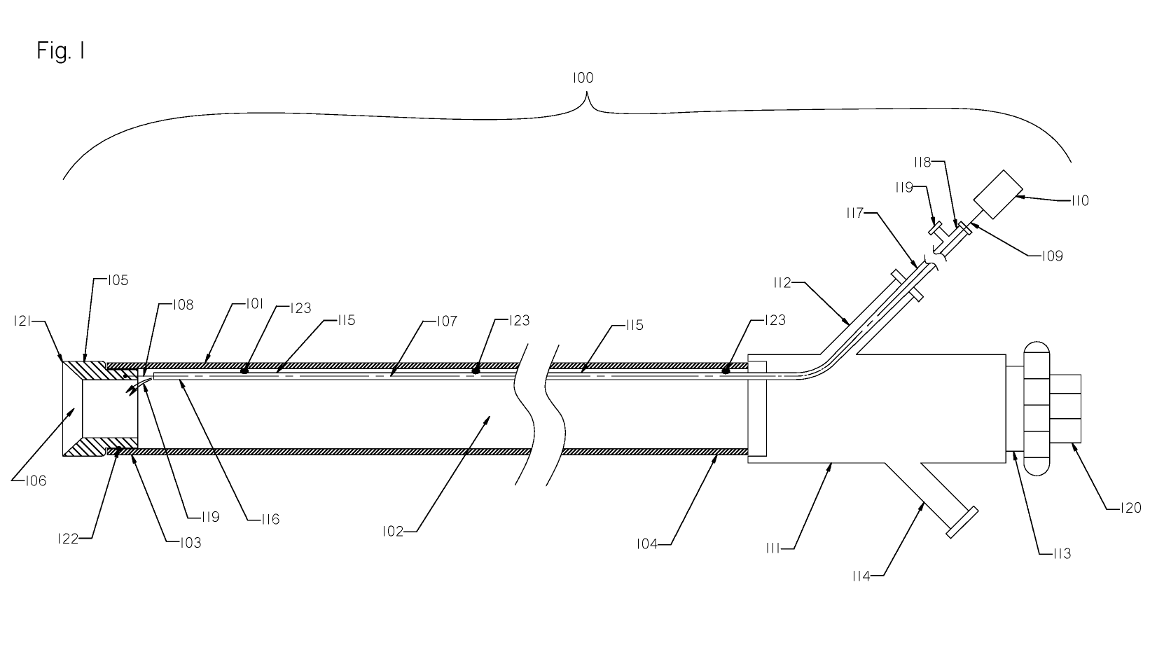

FIG. 1 shows an overview of the lead extraction device 100 of the present invention. The lead extraction device 100 comprises a shaft 101 having a lumen 102, a distal end 103 and a proximal end 104. A tubular metallic tip/ring 105 or tip having an inner aperture 106 is positioned on the distal end 103 of the shaft 101 and aligned coaxially with the lumen 102 of the shaft 101. A vibrational wire 107 having a distal end 108 and a proximal end 109 is extended longitudinally within the lumen 102. The distal end 108 of the wire 107 is attached to the tip/ring 105 while the proximal end 109 of the wire 107 is extended outside the shaft 101 and provides connection to a vibrational energy source 110.

A dual arm Y-connector 111 is attached to the proximal end 104 of the shaft 101. The dual arm Y-connector 111 comprises a vibrational energy delivery port 112, a lead exit port 113 and a suction port 114. A secondary lumen 115 having a distal end 116 and a proximal end 117 is extended within the shaft 101 and houses the wire 107. The distal end 116 of the lumen 115 is terminated at the vicinity of the tip 105. The lumen 115 comprises a side connector 118 to receive cooling irrigant 119 that is to be delivered along the wire 107 during the delivery of vibrational energy from the vibrational energy source 110 to the tip/ring 105. The cooling irrigant 119 exits at the distal end 116 of the lumen 115.

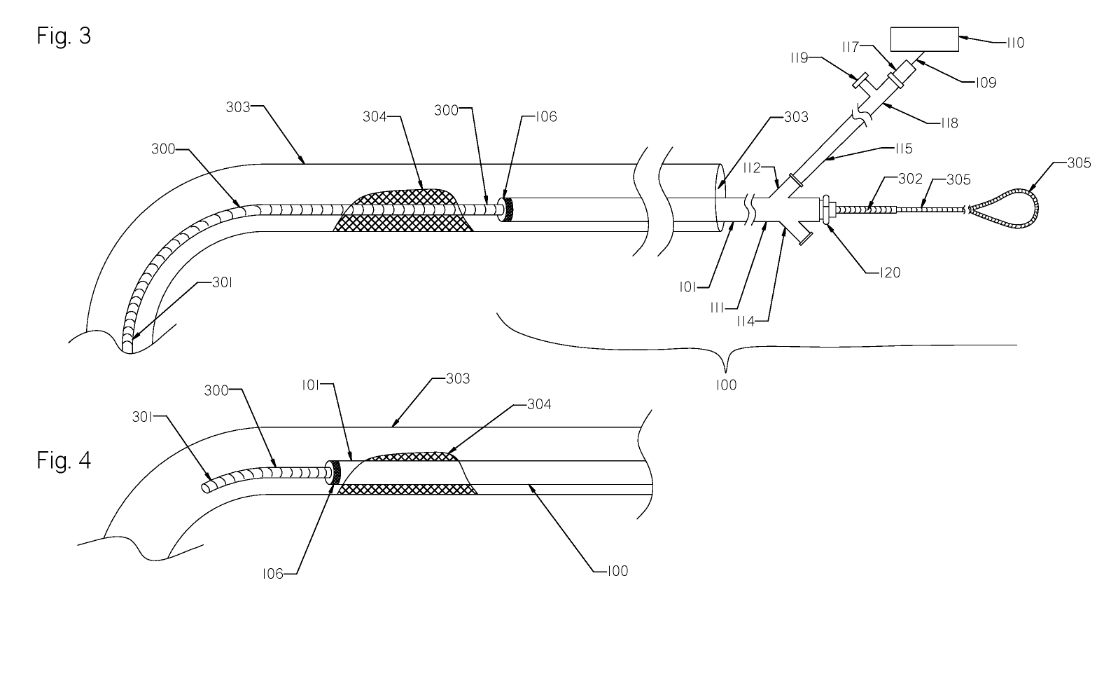

The lead port 113 of the dual arm Y-connector is constructed as the exit for lead during lead removal. The suction port 114 of the dual arm Y-connector 111 is designated for aspiration of cooling irrigant 119 and any tissue or debris created during the lead removal and as described and shown in FIG. 3. The suction port 114 may be connected to any aspiration pump or any suitable syringe. A Tuohy-Borst adapter 120 is attached to the lead exit port 113 of the dual arm Y-connector 110 and provides an internal seal to aspirate cooling irrigant 119 and debris of cut tissue while proceeding with lead removal.

The shaft 101 of the lead removal device 100 may a flexible, pushable, and torqueable elongate body such that it is configured to be slid though the vasculature of the patient and around lead placed inside the body. The shaft 101 may be composed of a polymer or flexible metallic alloy and may include a plurality of slits along its surface for improving flexibility.

The tubular tip/ring 105 disposed at the distal end 102 of the shaft 101 is configured to mechanically cut tissue as it is pushed distally and/or when it is pushed and rotated. In addition, the tubular tip/ring 105 which is attached to the wire 107 may be vibrated by vibrational energy delivered via the wire 107 from the vibrational energy source 110 to the tip/ring 105. The tip/ring 105 is made of metal or metal alloy including but not limited to SST, Titanium, Aluminum, Nitinol and other similar materials. The wire 107 may be made of metal or metal alloys including SST, Titanium, Nitinol and other similar materials. Two or more vibrational wires 107 may be attached to the tubular tip/ring 105 if needed (not shown).

The cutting tip/ring 105 is circumferential and may include a serrated or sharp continuous edge 121 which functions as a cutting element when it is pushed and/or rotated and/or vibrated. The tip/ring 105 may define an outer diameter that is the same as, or similar to, the outer diameter of the shaft 101 so as to be flush with the outer surface of the shaft 101.

During the delivery of vibrational energy to the cutting tip/ring 105, the tip/ring 105 will vibrate or move back and forth at the assigned frequency. It is desirable that longitudinal displacement of the cutting tip/ring 105 is more than 5 microns (pick to pick). Such minimum displacement of the cutting tip/ring 105 is needed to provide a minimum ablation efficacy required to further facilitate separation of lead encapsulating tissue.

The wire 107 is attached in an off-centered manner with respect to the cutting tip/ring 105, thereby allowing the removed lead to be placed through the inner aperture 106. The saline cooling irrigant 119 is delivered into the port 118 located at the proximal end 117 of the lumen 115 and serves for cooling the vibrational wire during delivery of vibrational energy from the vibrational energy source 110 to the cutting tip/ring 105. The cooling irrigant exits at the distal end 116 of the lumen 115. The saline cooling irrigant 119 also helps flush removed tissue and is aspirated via the aspiration port 114 outside the body.

The cutting tip/ring 105 can be configured with a smaller-diameter proximal region 122 that is stepped down from a distal region that includes the distal edge 121. The cutting tip/ring 105 may be affixed to the distal end 103 of the shaft 101 at the proximal region 122 by any suitable methods including but not limited to glue bonding, fusing and other similar techniques. Also, the cutting tip/ring 105 may be at least partially located within the distal end 103 of the shaft 101, and allowed to be free floating or vibrating, during the delivery of vibrational energy.

The secondary lumen 115 is extended longitudinally within the shaft 101. The vibrational wire 107 is extended inside the lumen 115. The lumen 115 is at least partially positioned at an off-centered location within the shaft 101. The lumen 115 may be attached internally at one or more locations 123 to the shaft 101 to avoid any blocking of inner lumen 102 of the shaft 101, and to minimize potential interference while introducing leads inside the inner lumen 102 during lead removal as described in FIG. 3.

The secondary lumen 115 may in one embodiment comprise a tubing inside the shaft 101. In another embodiment, a multi-lumen shaft can be provided where a first lumen serves as functions of the inner lumen 102, and a second lumen serves the function of the secondary lumen 115 function.

The off-centered positioning of the vibrational wire 107 within the shaft 101 allows for the provision of a larger or more spacious inner lumen 102, so that the inner lumen 102 can more effectively receive leads while maintaining the smallest overall outer diameter OD possible. Off-centered attachment of the vibrational wire 107 to the tip 105 also preserves the largest possible opening 106 within the tip 105. Also, placement of the secondary lumen 115 over the vibrational wire 107 in an off-centered arrangement allows cooling irrigant 119 to exclusively surround the vibrational wire instead of filling the entire inner lumen 102 of the shaft 101.

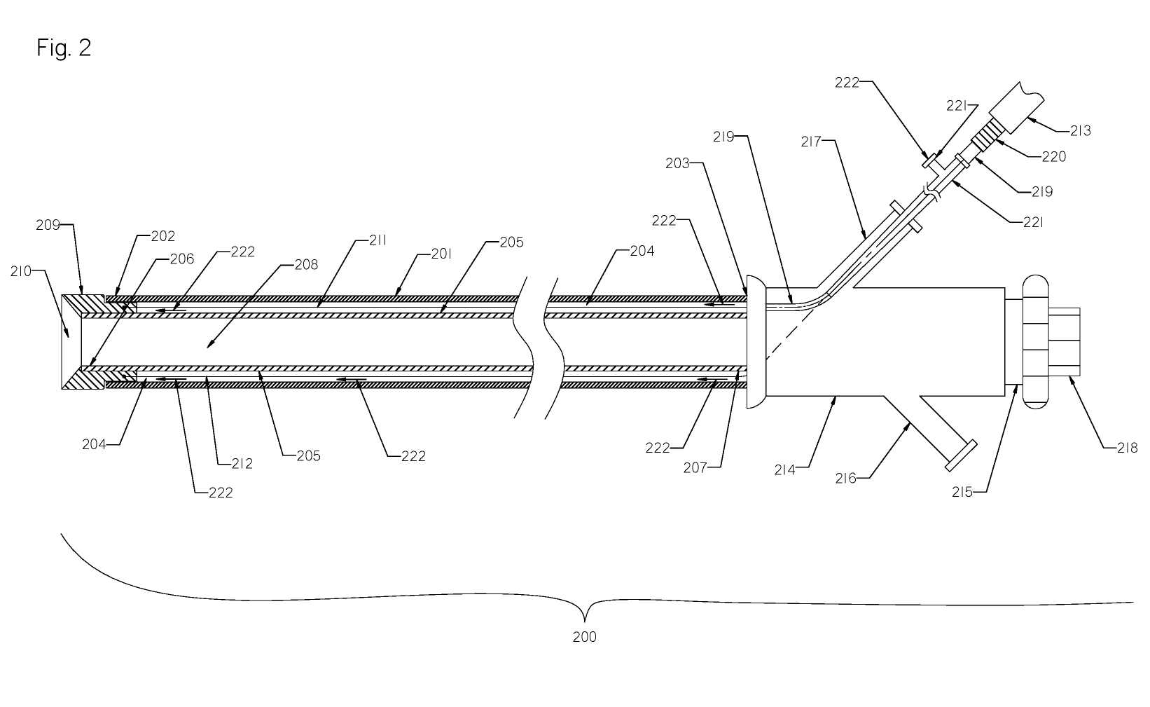

FIG. 2 shows an alternative version of a lead extraction device 200 comprising an outer shaft 201 having a distal end 202, a proximal end 203, and a longitudinally extending lumen 204. An inner shaft 205 has a distal end 206, a proximal end 207 and a lumen 208 extending longitudinally therethrough. The inner shaft 205 is positioned inside the lumen 204 of the outer shaft 201. A tubular metal tip/ring 209 having a circumferential aperture 210 is disposed at the distal end 202 of the outer shaft 201 and at the distal end 206 of the inner shaft 205. The tip/ring 209 is aligned coaxially with the inner lumen 208 of the inner shaft 205 such that it is suitable to receive lead to be extracted into the aperture 210 and along the lumen 208 of the inner shaft 205.

Two vibrational wires 211 and 212 are extended between the inner shaft 205 and the outer shaft 201. The wires 211 and 212 are attached to the tip/ring 209 on one end and to a vibrational energy source 213 on the other end (not shown). Multiple numbers of vibrational wires may be disposed between the inner shaft 205 and the outer shaft 201 and attached in a similar fashion if needed. If multiple wires are used, it is preferable that these wires are spaced in a symmetrical fashion: for example, if two wires are used, these wires should be at the opposite sides, approximately 180 degrees apart. If three wires are used, it is preferable to space them apart by 120 degrees, and so on.

The tip/ring 209 can be configured with a smaller-diameter proximal region that is stepped down from a distal region. The tip/ring 209 may be free floating while positioned within the distal end 202 of the shaft 201 and on the distal end 206 of the inner shaft 205. Alternatively, the tip/ring 209 may be affixed to the distal end 202 of the outer shaft 201 while free floating on the distal end 206 of the inner shaft 205 (not shown). Furthermore, the tip/ring 209 may be affixed to the distal end 206 of the inner shaft 205 while free floating on the distal end 202 of the outer shaft 201 (not shown). Also, the tip/ring 209 may be affixed to both distal ends 206 and 202 of the inner shaft 205 and outer shaft 201, respectively (not shown).

The proximal end 203 of the outer shaft 201 is attached to a dual Y-connector 214 having a lead exit port 215, an aspiration port 216 and a vibrational wire exit port 217. In addition, a Touhy-Borst adapter 218 is attached to the lead exit port 215 to provide an internal seal to aspirate debris and tissue cut while proceeding with lead removal.

A tubing 219 is positioned within the dual Y-connector 214 and extends outside the dual Y-connector 214 to house the wires 211 and 212, and is attached to the sonic connector 220. The sonic connector 220 attaches both wires 211 and 212 to the vibrational energy source 213. The additional side connector 221 is placed distally on the tubing 219 to provide an irrigation entry 222. The cooling irrigant introduced through the entry 222 is infused into the port 221 and is delivered between the inner shaft 205 and the outer shaft 201 towards the tip/ring 209. The irrigation exits near the tip/ring 209 and may be aspirated into the inner lumen 205 towards the aspiration port 216 and outside the body. Alternatively, the tip/ring 209 may include radial holes (not shown) to allow the irrigant to exit the tip/ring 209 into the aperture 210 and be further aspirated into the inner lumen 205.

The shape and size of the lead removal devices 100 and 200, as well as the tips/rings 105 and 209 shown in FIGS. 1 and 2 are merely exemplary, and are not limited to any particular sizes, dimensions, configurations or shapes shown.

FIG. 3 is a schematic view of a pacemaker lead 300 having a distal end 301 and a proximal end 302, and located in the vein 303 of a human being. The very distal and terminating electrode of the lead 300 is anchored distally to the ventricular heart chamber (not shown). The lead 300 is enclosed within the encapsulated tissue 304 to the wall of the vein 301. Such encapsulating tissue 304 often makes it difficult to apply conventional techniques for removing the lead 300.

At the beginning of the lead removal procedure, the lead 300 is detached, cut from the pacemaker or defibrillator device (not shown). An initial extraction of the lead 300 can be performed by simply pulling the proximal end 302 of the lead 300. The entire body of the lead 300 can also be rotated to facilitate lead retraction. If the lead 300 does not move freely, a locking stylet 305 (e.g., Liberator Universal Locking Stylet, Cook Medical, Bloomington, Ind.) can be used to aid with traction. Manual pulling of the lead 300, including use of the locking stylet, are well known in the art and will not be further described.

If such manual approaches in pulling the lead 300 and using the stylet 305 are not successful, the lead removal device 100 is inserted over the locking stylet 305 and the lead 300, and positioned proximal to the encapsulating tissue 304. Using the cutting ring 105 having aperture 106 and pushing the distal end 103 of the lead removal device 100 against the encapsulating tissue 304 may separate the lead 300 from the encapsulating tissue 304.

Activation of vibrational energy from the energy source 110 will deliver vibrations via the proximal end 109 of the wire 107 (not shown) to the tip/ring 105 to further enhance separation and liberation of the lead 300 from the encapsulating tissue 304. During the ablation of the encapsulating tissue 304 with the vibrating tip/ring 105, additional approaches to separate the lead 300 from the encapsulating tissue 304 may be undertaken by further pulling the lead 300 proximally and outside the encapsulating tissue 304.

Suction may be attached to the port 114 to aspirate the cut encapsulating tissue 304 and other created tissue residuals outside the body via the port 114.

FIG. 4 illustrates the cutting tip/ring 105 and the distal end 103 of the lead extraction device 100 advanced distally through the encapsulating tissue 304. The lead 300 is separated from the encapsulating tissue 304 and the distal end 301 of the lead 300 is separated from the ventricular heart chamber.

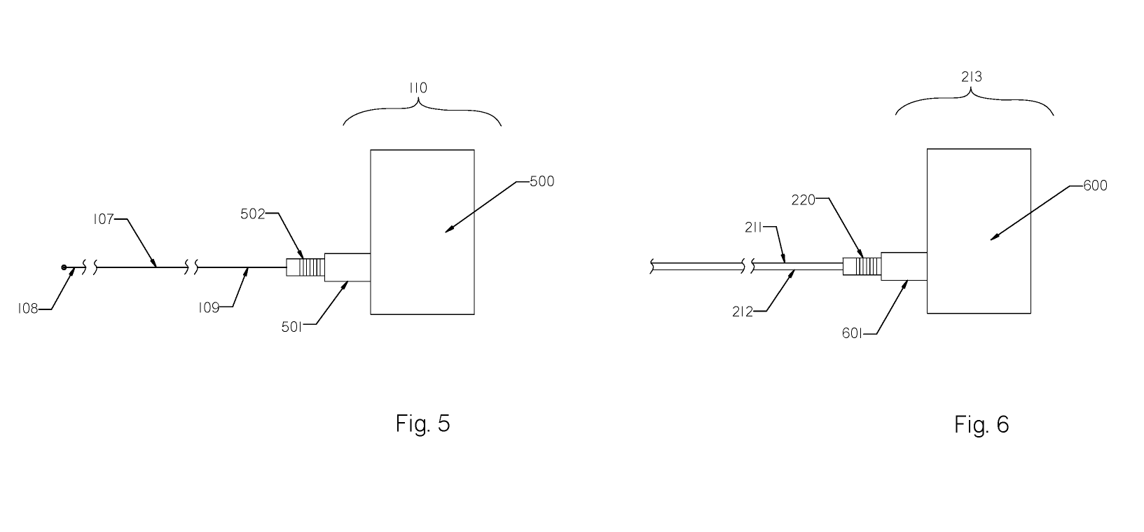

FIG. 5 illustrates an ultrasound energy source 110 as shown in FIGS. 1 and 3. The energy source 110 includes an ultrasound generator 500 and a piezoelectric transducer 501, both located outside the human body. The ultrasonic generator 500 converts electrical power into high frequency current to drive the ultrasonic transducer 501. The transducer 501 is made up of piezoelectric crystals and other active elements which expands and contracts when excited with a high frequency current, thus converting electrical energy to vibrations.

The proximal end 109 of the vibrational wire 107 is attached to the transducer 501 via a sonic connector 502. The transducer 501 generates ultrasonic activity that produces vibrations and sound waves which are propagated from the transducer 501 via the sonic connector 502 into the proximal end 109 of the vibrational wire 107 to its distal end 108 that is attached to the tip 105 as shown in FIG. 1.

FIG. 6 illustrates an ultrasound energy source 213 as shown in FIG. 2. The energy source 213 is similar to the energy source 110 shown in FIGS. 1 and 3 and includes an ultrasound generator 600 and a piezoelectric transducer 601. The vibrational wires 211 and 212 are encapsulated together proximally within the sonic connector 220 which attaches both wires 211 and 212 to the piezoelectric transducer 601.

The methods described herein employ a tissue cutting and ablation process that cuts a surrounding tissue during the delivery of vibrational energy to the tip/ring 105 and tip/ring 209. When vibrational energy is delivered at a frequency range of 1 Hz to less than 1 MHz and at a power below 20 watts to tissue, plaque or calcifications, vibrational energy will transiently disrupt the integrity of the plaque and the cell membranes without creating permanent damage to the vessel wall, or to soft and healthy surrounding tissue.

In a typical embodiment of the invention, vibrating tip 105 or 209 is in contact with or in proximity to a treatment area within the vessel using a vibrational energy frequency of about 1 Hz to less than 1 MHz, preferably 1 kHz-100 kHz, and a power of less than about 20 watts is used to ablate scarred and calcific tissue. Power levels above 20 watts may cause permanent damage to the vessel wall such as thermal damage, necrosis and vessel rupture or perforations when vibrational energy is delivered by the lead removal device.

Vibrational energy is produced by the transducers 501 in FIG. 5 and 601 in FIG. 6 located outside the body and propagated via the vibrational wire 108 in FIGS. 1 and 3, and the wires 211 and 212 in FIG. 2, in form of longitudinal waves to the tip 105 in FIG. 1 and the tip 209 in FIG. 2. Other ultrasound wave forms may be included such as surface waves and transverse waves but are less relevant in propagating ultrasound energy to generate needed displacement at the tip 105 in FIG. 1 and the tip 209 in FIG. 2.

As used herein, “power” of the lead removal device delivering vibrational energy refers to watts of power delivered at the distal end of the tip/ring 105 and the tip/ring 209.

Some theoretical considerations have been provided herein as to the mechanisms by which these therapeutic methods are effective; these considerations have been provided only for the purpose of conveying an understanding of the present invention, and do not limit the scope of the claims herein.

It will be appreciated by persons skilled in the art that the present invention is not limited to what has been particularly shown and described herein above. It should be noted that the accompanying drawings are not to scale. A variety of modifications and variations are possible in light of the above teachings without departing from the scope and spirit of the invention, which is limited only by the following claims:

1. A lead extraction device comprising:
an ultrasound energy source comprising an ultrasound transducer and a generator located outside the body;
a shaft having a distal end, a proximal end and a lumen extending longitudinally through the shaft;
a tip disposed at the distal end of the shaft having a circumferential aperture that is aligned off-center with respect to the lumen of the shaft, wherein the shaft and the tip are adapted to receive lead to be extracted;
a sonic connector;
a vibrational wire for propagating ultrasound energy having a distal end and a proximal end, wherein the distal end of the vibrational wire is attached to the tip at an off-centered location within the lumen of the shaft, wherein the proximal end of the vibrational wire is attached to the ultrasound transducer via the sonic connector;
a secondary lumen extending longitudinally within the shaft, wherein the vibrational wire extends inside the secondary lumen and is positioned at least partially at an off-centered location within the lumen of the shaft, and
wherein coolant irrigation is provided through the secondary lumen during the delivery of ultrasound energy.

2. The lead extraction device of claim 1, wherein the tip has a distal end and a proximal end, wherein the vibrational wire is attached to the tip, wherein the proximal end of the tip is affixed to the shaft, and wherein the tip undergoes longitudinal displacement pick to pick of more than 5 microns.

3. The lead extraction device of claim 1, wherein the tip has a distal end and a proximal end; wherein the vibrational wire is attached to the tip; wherein the proximal end of the tip is at least partially positioned within the distal end of the shaft but not affixed to the shaft, and wherein the tip undergoes longitudinal displacement pick to pick of more than 5 microns.

4. The lead extraction device of claim 1, wherein two or more vibrational wires are attached to the tip.

5. The lead extraction device of claim 1, wherein the vibrational wire propagates ultrasound energy from the transducer to the tip in form of longitudinal waves.

6. The lead extraction device of claim 1, wherein the secondary lumen is at least partially located within the shaft and affixed to the shaft.

7. The lead extraction device of claim 1, wherein vibrational wire propagates ultrasound energy from the transducer to the tip at frequency between 1 kHz to 100 kHz, and wherein the power at the tip is less than 20 Watts.

8. The lead extraction device of claim 1, wherein at least one lead is extended within the shaft during delivery of ultrasound energy.

9. A lead extraction device comprising:
an ultrasound energy source comprising an ultrasound transducer and a generator located outside the body;
a shaft having a distal end, a proximal end and a lumen extending longitudinally through the shaft;
a tip disposed at the distal end of the shaft having a circumferential aperture that is aligned off-center with respect to the lumen of the shaft, wherein the shaft and the tip are adapted to receive lead to be extracted;
a sonic connector;
a vibrational wire for propagating ultrasound energy having a distal end and a proximal end, wherein the distal end of the vibrational wire is attached to the tip at an off-centered location within the lumen of the shaft, wherein the proximal end of the vibrational wire is attached to the ultrasound transducer via the sonic connector;
a secondary lumen extending within the shaft and positioned off-centered inside the lumen of the shaft, wherein the vibrational wire extends outside the secondary lumen in an off-centered location within the lumen of the shaft, and
wherein coolant irrigation is provided inside the shaft and outside the secondary lumen during ultrasound energy delivery.

10. The lead extraction device of claim 9, wherein the tip is positioned in one of the following manners: the tip is affixed to the inner shaft, the tip is affixed to the outer shaft, the tip is affixed to both the inner and outer shafts, and the tip is free floating within both the inner and outer shafts.
